(19) United States
(12) Patent Application Publication
(10) Pub. No.: US 2005/0084846 A1
(43) Pub. Date: Apr. 21, 2005

(54) Method for predicting development of interstitial pneumonia by quantifying MUC-1

(76) Inventors: Hiroaki Okamoto, Shimotsuga-gun (JP); Hajime Tokita, Tokyo (JP)

Correspondence Address: CHOATE, HALL & STEWART LLP, EXCHANGE PLACE, 53 STATE STREET, BOSTON, MA, 02109, US

(21) Appl. No.: 10/785,891

(22) Filed: Feb. 24, 2004

(30) Foreign Application Priority Data
Oct. 15, 2003 (JP) 2003-355727

(51) Int. Cl. C12Q 1/70; C07K 16/24

(52) U.S. Cl. 435/5; 530/388.23

(57) ABSTRACT

Development of interstitial pneumonia due to administration of a drug can be predicted by quantifying MUC-1 in a body fluid; and determining that a possibility for the development of interstitial pneumonia is high when the amount of MUC-1 exhibits a value higher than a normal value.

### RELATED APPLICATIONS

This application claims the benefit of Japanese Patent Application No. 2003-355727, filed on Oct. 15, 2003, the entire teachings of which are incorporated herein by reference.

### FIELD OF THE INVENTION

The present invention relates to a method for predicting development of interstitial pneumonia due to drug administration by quantifying MUC-1, and a reagent for quantifying MUC-1 antigen used in the method.

### DESCRIPTION OF THE RELATED ART

Interferon α is widely used for the treatment of chronic hepatitis C. Although infrequent, severe interstitial pneumonia has been reported as a side effect of interferon α (Moriya K, Yasuda K, Koike K, et al.: Induction of interstitial pneumonitis during interferon treatment for chronic hepatitis C. J Gastroenterol 29: 514-517, 1994). The interstitial pneumonia progresses as far as the administration of interferon continues. Thus, it is desired to observe a patient carefully from the early stage of the administration of interferon (Iino S, Hino K, Yasuda K: Current state of interferon therapy for chronic hepatitis C. Intervirology 37: 87-100, 1994).

The quantification of LDH in serum has been used to complement the diagnosis of interstitial pneumonia. However, the LDH nonspecifically increases in many disorders, so that it is inappropriate as a marker for the interstitial pneumonia. In recent years, a protein specific to a pulmonary epithelial tissue, such as a KL-6 antigen (hereinafter, referred to as MUC-1, Khono N, Akiyama M, Kyoizumi S, Honda M, Kobuke K, Yamakido M: Detection of soluble tumor-associated antigens in sera and effusions using novel monoclonal antibodies, KL-3 and KL-6, against lung adenocaricinoma. Jpn J Clin Oncol 18: 203-216, 1997), and hydrophobic surfactant proteins A (Kuroki Y, Tsutahara S, Shijubo N, et al.: Elevated levels of lung surfactant protein A in sera from patients with idiopathic pulmonary fibrosis and pulmonary alveolar proteinosis. Am Rev Resir Dis. 147: 723-729, 1993) and D (Honda Y, Kuroki Y, Matsuura E, et al.: Pulmonary surfactant protein D in sera and bronchoalveolar lavage fluids. Am J Respir Crit Care Med 152: 1860-1866, 1995) (referred to as SP-A and SP-D, respectively) have become used for the diagnosis of interstitial pneumonia. It is thought that serum level of these proteins specific to the pulmonary epithelial tissue increases because interstitial pneumonia causes an increase of permeability between a blood vessel and air in the lung (Khono N: Serum marker KL-6/MUC-1 for the diagnosis and management of interstitial pneumonitis. J Med Invest 46: 151-158, 1999).

The MUC-1 is a high molecular weight mucin glycoprotein strongly expressed in type II alveolar epithelial cells and bronchial epithelial cells (Khono N et al., Jpn J Clin Oncol 18: 203-216, 1997). An increase of MUC-1 in serum is observed in 70-100% of patients of interstitial pneumonia, hypersensitivity pneumonia, sarcoidosis, and radiation pneumonitis, but not observed in patients of non-interstitial lung disease (Khono N, J Med Invest 46: 151-158, 1999). In addition, it is reported that the quantitative value of MUC-1 in blood is useful for foreseeing idiopathic pulmonary fibrosis (Yokoyama A, Khono N, Hamada H, et al.: Circulating KL-6 predicts the outcome of rapidly progressive idiopathic pulmonary fibrosis. Am J Respir Crit Care Med 158: 1680-1684, 1998).

It is reported that the SP-A and SP-D are expressed in nonciliary epithelial cells of peripheral airway, Clara cells and type II alveolar epithelial cells, and the quantification of SP-A and SP-D in serum is useful for the diagnosis and prediction of idiopathic interstitial pneumonia (Kuroki Y et al. Am Rev Resir Dis. 147: 723-729, 1993, Honda Y et al. Am J Respir Crit Care Med 152: 1860-1866, 1995, and Takahashi H, Fujishima K, Koba H, et al.: Serum surfactant protein A and D as prognositic factors in ideopathic pulmonary fibrosis and their relationship to disease extent. Am J Respir Crit Care Med 162: 1109-1114, 2000).

However, there is still no marker known in the art, which can predict interstitial pneumonia induced by the administration of interferon.

### SUMMARY OF THE INVENTION

An object of the present invention is to develop a method and a reagent for predicting development of interstitial pneumonia due to administration of interferon.

The inventors of the present invention considered that a patient who develops interstitial pneumonia due to the administration of interferon has already developed latent interstitial pneumonia before the administration as a result of an immune response to hepatitis C virus or the like, and that the development of interstitial pneumonia occurs as the interferon activates the immune system. Furthermore, they considered that the development of interstitial pneumonia due to the administration of interferon could be predicted if the latent interstitial pneumonia is found by the quantification of MUC-1 excellent in both sensitivity and specificity.

The inventors of the present invention have completed the present invention by finding out that the serum level of MUC-1 in a patient who develops interstitial pneumonia due to the administration of interferon already exhibits a large value exceeding a normal value even before the administration, and that the quantification of the MUC-1 allows the development of interstitial pneumonia to be predicted.

That is, the present invention relates to the following:
(1) A method for predicting development of interstitial pneumonia due to administration of a drug, comprising: quantifying MUC-1 in a body fluid; and determining that a possibility for the development of interstitial pneumonia is high when the amount of MUC-1 exhibits a value higher than a normal value.
(2) The method according to (1), wherein the drug is interferon.
(3) The method according to (2), wherein the interferon is interferon α.
(4) The method according to any one of (1) to (3), wherein MUC-1 is quantified by an immunochemical method.
(5) The method according to (4), wherein the immunochemical method is an immunochemical method using KL-6 antibody.
(6) A reagent for predicting development of interstitial pneumonia due to administration of a drug, comprising an antibody against MUC-1.
(7) The reagent according to (6), wherein the antibody against MUC-1 is KL-6 antibody.

### BRIEF DESCRIPTION OF DRAWINGS

FIG. 1 shows quantified values of MUC-1 (KL-6), LDH, SP-A, and SP-D before the administration of interferon and after the development of interstitial pneumonia, for seven cases in which interstitial pneumonia was developed due to the administration of interferon.

FIG. 2 shows comparison of the quantified value of MUC-1 (KL-6) before the administration of interferon between the cases in which interstitial pneumonia was developed due to the administration of interferon and the cases without the development thereof.

### DESCRIPTION OF THE PREFERRED EMBODIMENTS

Hereinafter, embodiment of the present invention will be described in detail.

The term “body fluid” used herein includes blood, plasma, serum, pulmonary lavage fluid, and the like which are obtained from the living body.

In the present invention, it is found that latent interstitial pneumonia that has been developed may become interstitial pneumonia as triggered by administration of a drug. Therefore, the drug to be administered is not limited to interferon, but preferable is a drug that affects an immune response, more preferable is interferon, and further more preferable is interferon-α.

The term “MUC-1” refers to a high molecular weight mucin glycoprotein strongly expressed in type II alveolar epithelial cells and bronchial epithelial cells (Khono N et al., Jpn J Clin Oncol 18: 203-216, 1997) and is identified as antigens to be reacted with monoclonal antibodies including Ma552, Ma695, DF3, and FH6 (Tumor Biol; 19 (Suppl), 134-146, 1998) and KL-6 (Int. J. Cancer; Supplement 8, 81-83, 1994).

Examples of the monoclonal antibodies known in the art, which recognize MUC-1 antigen, include Ma552, DF3, and FH6 (Tumor Biol; 19 (Suppl 1), 134-146, 1998) and KL-6 (Int. J. Cancer; Supplement 8, 81-83, 1994). They can be prepared by the methods described in the above documents. In addition, Ma552 and DF3 are commercially available from Cosmo Bio Co., Ltd. and DAKO Co., Ltd, respectively.

KL-6 antibody referred to herein is one described in Japanese Patent No. 2011158.

Among the monoclonal antibodies that recognize MUC-1 antigens, preferable is KL-6 antibody. A method for preparing KL-6 antibody is also described in Japanese Patent No. 2011158.

Cups or beads coated with KL-6 antibody, and enzyme- or ruthenium (Ru)-labeled KL-6 antibody can be purchased from Sanko Junyaku Co., Ltd. under the name of Eitest KL-6 or Picolumi KL-6. In this specification, “U/ml” is defined in accordance with the definition in Eitest KL-6.

Examples of the method for measuring MUC-1 may include an immunochemical method, electrophoresis, chromatography, or the like. As an electrophoresis method, there is a method for detecting cytochrome as a band by carrying out a polyacrylamide electrophoresis, a method for detecting it as a peak by a capillary electrophoresis, or the like. In addition, as for chromatography method, there is a method for detecting its peak in high performance liquid chromatography. In some case, labeling with fluorescence may be allowed for raising the sensitivity. However, the present invention is not limited to those examples.

The method for quantifying MUC-1 is preferably an immunochemical method from the viewpoints of sensitivity and simplicity. Here, the term “immunochemical method” means a method for quantifying MUC-1 using antibodies against MUC-1. Examples of the immunochemical method include various kinds of methods, such as a competitive method in which MUC-1 is labeled, a sandwich method in which an antibody is labeled, and a latex-beads method in which the aggregation of beads coated with an antibody is observed. Any method may be used in the present invention as far as it uses antibody against MUC-1. The antibody may be a monoclonal antibody or a polyclonal antibody. Examples of the labeling method include various kinds of methods, such as labeling with a radioactive isotope, labeling with a compound that emits electrochemical luminescence, labeling with fluorescence, labeling with enzyme, and labeling with biotin. However, the present invention is not limited to those examples. Methods for preparing and labeling the antibodies are described in, for example, “Sequel Biochemical Experimental Lecture 5, Immunobiochemical Research Techniques” (The Japanese Biochemical Society, Ed., published by Tokyo Kagaku Dozin Co., Ltd.) and “New Biochemical Experimental Lecture 12, Molecular Immunology III” (The Japanese Biochemical Society, Ed., published by Tokyo Kagaku Dozin Co., Ltd.).

As an example of the immunochemical method for quantifying MUC-1, the sandwich method will be described below in a stepwise manner.
(1) An antibody against MUC-1 is immobilized on beads or cups. The antibody to be immobilized may be a polyclonal antibody or a monoclonal antibody as far as it provides the beads or cups with desired sensitivity, that is, a sensitivity that enables quantification of preferably 100 U/ml, more preferably 50 U/ml of MUC-1. As an antibody, KL-6 antibody is preferable. The beads may be micro beads, in which case it is preferably magnetic micro beads. The immobilization may be achieved by bonding an antibody with a non-covalent bond or with a covalent bond. Typically, for covering non-specific binding sites on the beads or cups, a blocking operation is performed with a protein such as bovine serum albumin (BSA) or casein, and a surfactant such as Tween 20.
(2) Samples are diluted with a buffer containing a protein such as bovine serum albumin (BSA) or casein and a surfactant such as Tween 20 if required, and then added to the beads or cups. In addition, a known amount of MUC-1 is similarly diluted and added thereto.
(3) Preferably after washing the beads or cups with a buffer containing a surfactant such as Tween 20, a labeled anti-MUC-1 antibody preferably diluted with a buffer containing a protein such as bovine serum albumin (BSA) or casein and a surfactant such as Tween 20 is added. The labeled antibody may be a polyclonal antibody or a monoclonal antibody as far as it provides a desired sensitivity, that is, a sensitivity that enables quantification of preferably 100 U/ml, more preferably 50 U/ml of MUC-1. As an antibody, KL-6 antibody is preferable.
(4) After washing the beads or cups with a buffer containing a surfactant such as Tween 20, a measurement is performed according to the type of the label, i.e., the measurement is performed on radioactivity in the case of labeling with a radioactive isotope or on enzyme activity in the case of labeling with an enzyme. Alternatively, in the case of labeling with biotin, a labeled avidin is further added and the measurement is performed.
(5) The amount of MUC-1 contained in the sample is calculated by forming a calibration curve from the measurement values of a sample that contains the known amount of MUC-1.

Consequently, MUC-1 in the sample can be quantified by the steps described above.

Furthermore, the present invention also relates to a reagent for predicting the development of interstitial pneumonia due to the administration of interferon. As an example thereof, there can be mentioned a reagent for the quantification of MUC-1 in accordance with a sandwich method, which may contain (1) cups coated with an anti-MUC-1-antibody, or beads coated with an anti-MUC-1 antibody, or (2) a labeled-anti-MUC-1 antibody, and which may preferably further contain (3) a known concentration of a MUC-1 standard solution, (4) a diluent, and (5) a washing solution. Furthermore, in the case of labeling with an enzyme, (6) a coloring substrate and (7) a reaction-stopping solution may be included. These reagents can be purchased from Sanko Junyaku Co., Ltd. under the name of Eitest KL-6 or Picolumi KL-6.

The method and the reagent for quantifying MUC-1 disclosed in the present invention can be used for predicting the development of interstitial pneumonia due to the administration of interferon.

When the quantified value of MUC-1 measured by the method and the reagent for MUC-1 disclosed in the present invention is high and outside the distribution of the quantified values of MUC-1 of a patient who has not developed interstitial pneumonia (the upper limit of normal (UNL)), preferably a healthy adult, it is determined that the patient is very likely to develop interstitial pneumonia due to the administration of interferon. The value of UNL may be set as 500 U/ml, a value by which the patient is temporally diagnosed as interstitial pneumonia.

MUC-1 may serve as an index useful for precisely recognizing a possibility for development of interstitial pneumonia due to the administration of interferon.

### EXAMPLES

Hereinafter, the present invention will be described with reference to specific examples. However, the present invention is not limited to those examples.

### Example 1

### Backgrounds of Patients

The analyzed patients are 558 chronic hepatitis C patients provided with the administration of interferon-α from January 1992 to July 2000 in the alimentary division of Tokyo hospital of National Sanatoria. Among those, six patients (1.1%) developed interstitial pneumonia, which includes patients who received the administration of ribavirin and patients who did not receive the same. Among them, one patient (Case 5) developed interstitial pneumonia in 1993 during the administration of interferon-a, and after recovery, the patient received combined therapy of interferon-α-2b and ribavirin, and developed interstitial pneumonia again in 2002. During nine years before the development of interstitial pneumonia again, the patient did not surfer from a respiratory system disorder. Thus, the two developments were handled as two independent cases (5-1 and 5-2).

Diagnostic criteria for interstitial pneumonia developed by the administration of interferon are: 1) no respiratory system disorder is developed before the administration of interferon; 2) a symptom of the respiratory system is developed during the administration of interferon; 3) infiltrating shadows are found in the lower parts of both sides or over the whole area of the lung in chest CT and X-rays; 4) an abnormality is found in lung function and arterial blood gas analysis; and 5) no infectious disease is developed.

In Table 1, backgrounds of seven cases which developed interstitial pneumonia are shown. The anamnesis for the respiratory system was found in three out of seven cases, but no symptom of respiratory system was found at the time of initiating the administration.

TABLE 1

| | | | Anamnesis | | | | |
|---|---|---|---|---|---|---|---|
| | Age | | Blood | | | | |
| | (Years | | transfusion | | | | |
| Case | old) | Sexuality | history | Pulmonary disease | Allergy | Sho-saiko-to | Drinking history |
| 1 | 66 | Female | Age 36 | (—) | (—) | 2 months | (—) |
| 2 | 66 | Male | (—) | Tuberculosis (Age | (—) | 3 years | Opportunity |
| | | | | 22) | | | drinking |
| 3 | 54 | Female | (—) | (—) | Sedes, Kefral | (—) | (—) |
| 4 | 65 | Male | Age 26 | Tuberculosis (Age | (—) | (—) | Beer 400 ml/day |
| | | | | 26) | | | |
| 5-1 | 57 | Female | Age 22 | (—) | Antimicrobial drug | (—) | (—) |
| 5-2 | 66 | | | IP (Case 5-1) | | | |
| 6 | 54 | Male | Age 44 | (—) | Mackerel, Cold | (—) | Sake 3 go (go = 180 ml)/ |
| | | | | | remedy | | day |

In Table 2, the findings of the type of chronic hepatitis C virus, the quantity of virus RNA, AST and ALT test values, and liver tissue images are summarized.

TABLE 2

| | | HCV RNA | | | Liver |
|---|---|---|---|---|---|
| | HCV | titer | | | tissue |
| Case | genotype | (KIU/ml) | AST (IU/l) | ALT (IU/l) | image |
| 1 | 1b | 100 | 116 | 80 | F3/A3 |
| 2 | 1b | 220 | 121 | 74 | F4/A3 |
| 3 | 1b | 45 | 93 | 170 | F2/A2 |
| 4 | 1b | 64 | 52 | 66 | F1/A2 |
| 5-1 | 2b | 260 | 115 | 152 | F1/A2 |
| 5-2 | 2b | 843 | 315 | 467 | F1/A2 |
| 6 | 2b | 463 | 88 | 109 | F2/A2 |

In Table 3, the process of the treatment in each of the cases is summarized. In all cases, the treatment is canceled following with the development of interstitial pneumonia. An average total dosage of interferon is 427 MU and an average duration of the administration is 2.5 months.

TABLE 3

| | IFN | IP | | | Treatment/Exitus | |
|---|---|---|---|---|---|---|
| Case | Class | Total dosage | Onset | Primary symptom | Treatment | Exitus |
| 1 | r-α2a | 432 MU | 3.5 months | Unproductive cough, Pyrexia | Steroid | Improvement |
| 2 | natural α | 102 MU | 8 days | Unproductive cough, | Steroid | Death |
| | | | | Difficulty of breathing | | |
| 3 | r-α2a | 560 MU | 3.5 months | Unproductive cough, | (—) | Improvement |
| | | | | Difficulty of breathing | | |
| 4 | natural α | 980 MU | 5 months | Difficulty of breathing | Steroid | Improvement |
| | | | | during body movement | | |
| 5-1 | natural α | 168 MU | 1 month | Unproductive cough | (—) | Improvement |
| 5-2 | r-α2a + RIBA | 560 MU | 3 months | Unproductive cough | Steroid | Improvement |
| 6 | r-α2a + RIBA | 190 MU | 1 month | Unproductive cough, | Steroid | Improvement |
| | | | | Difficulty of breathing | | |

### Example 2

### Quantification of MUC-1 (KL-6), LDH, SP-A, and SP-D in Six Cases in Which Interstitial Pneumonia was Developed

In the following examples, MUC-1 value is quantified with KL-6 antibody and thus the MUC-1 value is described as KL-6. A serum sample was stored at −80° C. and used for the quantification described below.

The KL-6 in serum was quantified using commercially-available EIA kit (Eitest KL-6, manufactured by Sanko Junyaku Co., Ltd.). Similarly, the SP-A in the serum was quantified as described in the method reported in Kuroki Y et al. Am Rev Resir Dis. 147:723-729, and the SP-D was quantified using commercially-available EIA kit (SP-D EIA Kit, manufactured by Yamasa Corporation). The maximum values (UNLs) of the respective normal values are 500 U/ml for MUC-1, 43.8 ng/ml for SP-A, and 110 ng/ml for SP-D.

Furthermore, at that time, the calculation was performed under the conditions in which the UNL of LDH was 399 IU/I until September, 1993 and 474 IU/I thereafter according to the criteria of National Sanatoria Tokyo Hospital.

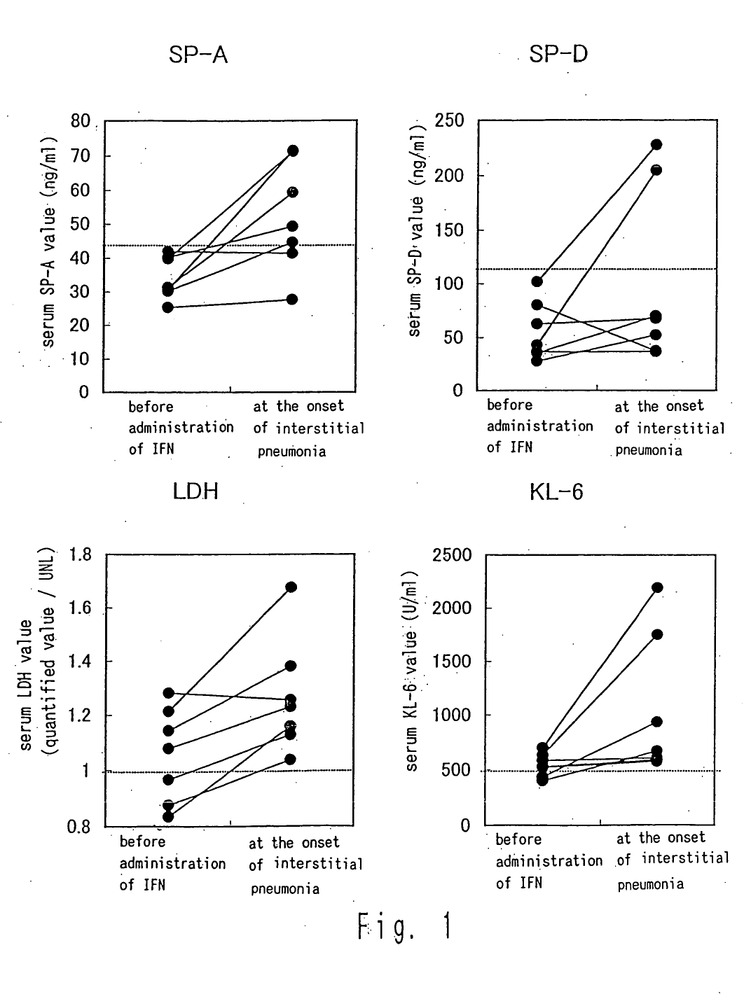

For seven cases in which interstitial pneumonia was developed due to the administration of interferon, the results of the quantifications of KL-6, LDH, SP-A, and SP-D before the interferon administration and after the development of interstitial pneumonia are shown in FIG. 1.

An appearance ratio of abnormally high value at the time of the development of interstitial pneumonia was KL-6: 100%, LDH: 100%, SP-A: 71%, and SP-D: 29%. An appearance ratio of abnormally high value before the administration of interferon was KL-6: 71%, LDH: 57%, SP-A: 0%, and SP-D: 0%. In five out of the seven cases in which interstitial pneumonia was developed due to the administration of interferon (five out of six cases when Case 5-2 as a recurrence case is excluded), KP-6 was found to exhibit an abnormally high value even before the administration.

### Example 3

### Comparison of KL-6 Values Before the Administration of Interferon Between Cases in Which Interstitial Pneumonia was Developed due to the Administration of Interferon and Cases Without the Development Thereof

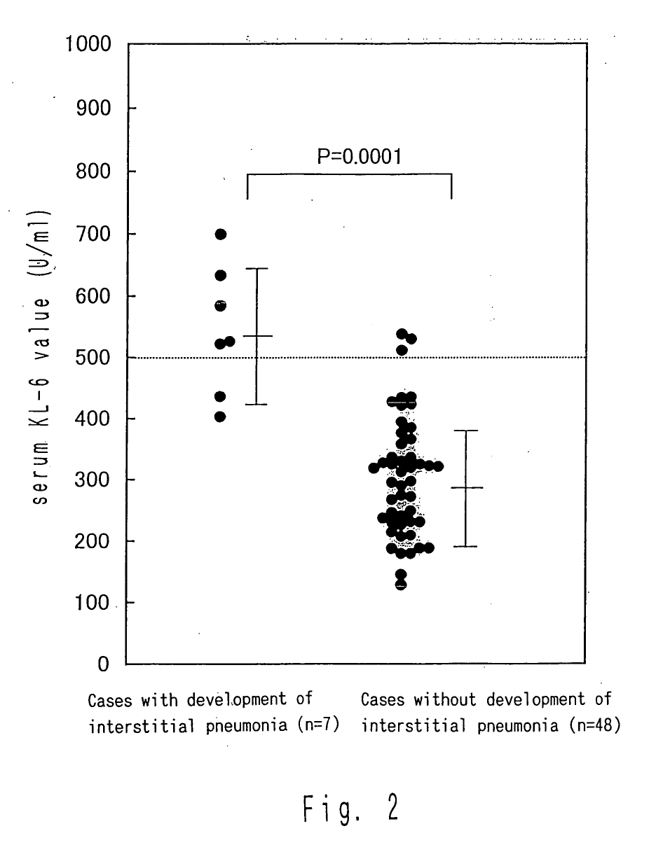

Of the cases in which interstitial pneumonia was developed due to the administration of interferon, many cases were observed in which KL-6 exhibited an abnormally high value even before the administration of interferon. Thus, from among patients not younger than the youngest patients (age 54 in Cases 3 and 6) who developed interstitial pneumonia, 48 chronic hepatitis C patients who did not develop interstitial pneumonia due to the administration of interferon were randomly sampled and subjected to the measurement of the KL-6 value before the administration. The results compared with those of the cases in which interstitial pneumonia was developed are shown in FIG. 2.

The abnormally high value was found in five out of seven cases (71%) with the development of interstitial pneumonia. On the other hand, the abnormally high value was found in only three out of 48 cases (6%) without the development of interstitial pneumonia. Thus, the appearance ratio of abnormally high value was significantly high in the cases with the development of interstitial pneumonia (P=0.0003). In addition, the KL-6 quantification value was 543±105 U/ml in the cases with the development of interstitial pneumonia and 304±98 U/ml in the cases without the development of interstitial pneumonia. Thus, the value was significantly high in the cases with the development of interstitial pneumonia (P=0.0001).

From the above results, it was suggested that the patient who developed interstitial pneumonia due to the administration of interferon had already developed latent interstitial pneumonia, and that there is a possibility that the interferon administration caused the latent interstitial pneumonia to develop into active interstitial pneumonia. It was shown that quantifying KL-6 before the administration of interferon is useful for predicting the development of interstitial pneumonia during the administration of interferon.

While the invention has been described in detail with reference to preferred embodiments thereof, it will be apparent to one skilled in the art that various changes can be made, and equivalents employed, without departing from the scope of the invention. Each of the aforementioned documents, including the foreign priority document, JP 2003-355727, is incorporated by reference herein in its entirety.

1. A method for predicting development of interstitial pneumonia due to administration of a drug, comprising: quantifying MUC-1 in a body fluid; and determining that a possibility for the development of interstitial pneumonia is high when the amount of MUC-1 exhibits a value higher than a normal value.

2. The method according to claim 1, wherein the drug is interferon.

3. The method according to claim 2, wherein the interferon is interferon α.

4. The method according to any one of claim 1, wherein MUC-1 is quantified by an immunochemical method.

5. The method according to claim 2, wherein MUC-1 is quantified by an immunochemical method.

6. The method according to claim 3, wherein MUC-1 is quantified by an immunochemical method.

7. The method according to claim 4, wherein the immunochemical method is an immunochemical method using KL-6 antibody.

8. A reagent for predicting development of interstitial pneumonia due to administration of a drug, comprising an antibody against MUC-1.

9. The reagent according to claim 8, wherein the antibody against MUC-1 is KL-6 antibody.
